(19) United States
(12) Patent Application Publication
(10) Pub. No.: US 2016/0157956 A1
(43) Pub. Date: Jun. 9, 2016

(54) METHOD AND DEVICE FOR CLEANING A SURGICAL INSTRUMENT

(71) Applicant: OLYMPUS WINTER & IBE GMBH, Hamburg (DE)

(72) Inventor: Dennis AEHLIG, Hamburg (DE)

(73) Assignee: OLYMPUS WINTER & IBE GMBH, Hamburg (DE)

(21) Appl. No.: 15/045,335

(22) Filed: Feb. 17, 2016

Related U.S. Application Data
(63) Continuation of application No. PCT/EP2014/002012, filed on Jul. 23, 2014.

(30) Foreign Application Priority Data
Aug. 21, 2013 (DE) 102013216532.3

(51) Int. Cl. A61B 90/70

(52) U.S. Cl. CPC A61B 90/70; A61B 2090/701

(57) ABSTRACT

A method for cleaning a surgical instrument with a cleaning fluid, the surgical instrument having at least one channel. The method including: applying a pressure to the cleaning fluid, flushing the channel by means of the cleaning fluid, and varying the pressure during the flushing.

### CROSS-REFERENCE TO RELATED APPLICATION

The present application is a continuation of PCT/EP2014/002012 filed on Jul. 23, 2014, which is based upon and claims the benefit to DE 10 2013 216 532.3 filed on Aug. 21, 2013, the entire contents of each of which are incorporated herein by reference.

### BACKGROUND

1. Field

The present application relates to a method for cleaning a surgical instrument having at least one channel, such as an endoscope, by means of a cleaning fluid. The present application furthermore relates to a device for cleaning a surgical instrument having at least one channel, such as an endoscope, by means of a cleaning fluid. The present application further relates to a use.

2. Prior Art

In the prior art, flexible endoscopes with internal channels are known that for example are used in gastrointestinal surgery. To clean such endoscopes, the internal channels are generally flushed with a cleaning medium under constant pressure with an unchanging volumetric flow.

To remove encrusted particles and particles jammed within the channel, for example a complicated pretreatment is carried out in which the endoscope, or respectively the channels, are manually brushed.

In this regard, methods have been developed in which air and water are alternatingly pumped through the channels. Furthermore, the so-called two-phase flow technique is known in which very fine droplets of fluid in a gas phase produce the same result as manual brushing.

The known methods for avoiding manual brushing have the disadvantage that corresponding cleaning devices must have additional components for the air supply and air passages and are correspondingly more complex and error-prone than simple flushing devices.

Furthermore, such prior art methods are only slightly suitable for removing so-called biofilms where there is an extensive and cohesive contamination of the endoscope channel. Removing such substances is infeasible, or is only feasible with very long processing times.

### SUMMARY

Based on this prior art, an object is to simplify the cleaning of an endoscope with an endoscope channel, such as improving the removal of a biofilm from an endoscope channel interior.

This object is achieved by a method for cleaning a surgical instrument having at least one channel, such as an endoscope, by means of a cleaning fluid, comprising the following procedural steps:

applying a pressure to the cleaning fluid,

flushing the channel by means of a cleaning fluid, and

varying the pressure during flushing.

Varying the pressure exerted on the cleaning fluid, or varying the pressure within the cleaning fluid, especially within the channel, such as in the endoscope channel, causes in particular deformations of the channel, whereby the channel expands when the pressure is increased and contracts when the pressure is reduced. By means of the deformations, contaminants adhering to the inner wall of the channel are released and can thereby be flushed more easily out of the channel with the cleaning fluid.

An advantage of such method is that biofilms and other extensive contaminants can also be released and flushed out.

Overall, the cleaning result is thereby improved, and the duration of cleaning is shortened.

In addition, another advantage of the shorter cleaning duration is that less cleaning fluid is used. This renders cleaning more economical and environmentally friendly.

The pressure can be repeatedly varied during flushing which further improves the cleaning effect.

Furthermore, the pressure can be varied repeatedly at regular intervals in time, which can be a periodic, cyclical or oscillating variation of the pressure. This has the advantage of technically easy implementation, such as being based on rotating drive machines with corresponding pressure actuators.

Pressure can be applied to the cleaning fluid using a cyclical pump device, which can be a pump device or a delivery apparatus which, due to the design, has several work cycles or work phases such as a suction cycle and a compression cycle, or an ejection cycle. A pulsating or oscillating pressure level can thereby be directly generated in the delivered or pumped cleaning fluid. Such a cyclical pump device ican be a reciprocating pump or a diaphragm pump.

A diaphragm pump has the further advantage that it does not have a brush seal which also makes a corresponding cyclical pump device suitable for delivering contaminated cleaning fluid.

Suitable diaphragm pumps are offered by KNF FLODOS AG, Sursee, Switzerland under the product name of NF 600 or NF 1.600, as well as by Eckerle lndustrie-Elektronik GmbH, Malsch, Federal Republic of Germany under the product name of CDP 6800, CDP 8800, DDP 5800 or DDP 550.

Checking the cleaning of the canal can comprises another procedural step of such method.

Checking the cleaning can involve a test of the extent to which cleaning was successful, or must be continued or repeated. The test can be carried out by determining a flow rate and/or by determining a pressure drop within the channel.

A constant pressure can be applied to the cleaning fluid during the cleaning check. In general, this allows more precise measuring results or cleaning results to be achieved.

The method can also be suitable for cleaning endoscope accessories and accessories for endoscope cleaning apparatuses, such as hoses, etc.

An underlying objective is furthermore achieved with a device for cleaning a surgical instrument having at least one channel, such as an endoscope, by means of a cleaning fluid, comprising a pump apparatus for the cleaning fluid and a connection, wherein the pump apparatus can be configured to apply a varying pressure to the cleaning fluid while flushing the channel.

To apply a varying pressure to the cleaning fluid, the pump apparatus of a cleaning device can comprise a cleaning apparatus, such as a cyclical pump device, such as a reciprocating pump or a diaphragm pump.

The channel to be cleaned can consist of a flexible material. The endoscope can be a flexible endoscope.

Alternatively, the pump apparatus can comprise a continuous pump device such as an automatic control device for changing an output pressure of the continuous pump device.

A continuous pump device can be a pump device or delivery device with a continuous, such as non-cyclical, delivery. A varying or variation in the pressure or output pressure of the continuous pump device can be achieved by varying the pump or delivery rate by means of the control device. This can be accomplished automatically.

A suitable continuous pump device can be a reciprocating pump with an electric drive operated by alternating current, wherein the control device can comprise a frequency converter which is located upstream from the electric drive for the alternating current. In this case by changing the frequency of the alternating current, the rotational speed of the reciprocating pump, and correspondingly its delivery rate and hence the output pressure, can be varied.

The pump apparatus can comprise at least two pump apparatuses, wherein one of the pump devices can be configured as a cyclical pump device, and another pump device can be configured as a continuous pump device.

The advantages of a continuous pump device, such as a high delivery rate per unit time is thereby synergistically combined with the advantages of a cyclical pump device, such as a design-based variation of the output pressure.

Furthermore, the volumetric capacity of the cleaning fluid and the variation of the pressure within the cleaning fluid, such as the amplitude and frequency of the pressure variation, can be set independent of each other in that the respective delivery rate of the continuous pump apparatus on the one hand, and the cyclical pump device on the other hand, are suitably adjusted.

The device can comprise a connection group with a plurality of connections for one channel each, such as an endoscope channel.

This further reduces the duration of cleaning a surgical instrument because a plurality of channels of the surgical instrument can be cleaned at the same time.

The device can comprise a plurality of connections or connection groups, wherein the pump apparatus can comprise at least one pump device which is exclusively assigned to one connection or connection group.

This yields great flexibility in operating the device because the pressure level of this connection, or respectively this connection group, can be set independent of other connections and connection groups by means of the pump device assigned exclusively to one connection or connection group.

A connection or connection group can comprise a control device for checking the cleaning of channels connectable to the connection or connection group, such as by means of determining a flow rate and/or by means of determining a pressure drop in the channel.

For this purpose, the control device can have a pressure sensor for determining a pressure of the cleaning fluid, and/or a flow sensor for determining the flow volume or flow rate.

An object is furthermore accomplished using a cyclical pump device, such as a diaphragm pump, and a continuous pump device, such as a reciprocating pump, for cleaning a channel of a surgical instrument, such as an endoscope channel.

Further characteristics will become apparent from the description of the embodiments together with the claims and the included drawings. Embodiments can fulfill individual characteristics or a combination of several characteristics.

### BRIEF DESCRIPTION OF THE DRAWINGS

The embodiments are described below, without restricting the general idea of the invention, using exemplary embodiments with reference to the drawings, whereby we expressly refer to the drawings with regard to all details that are not explained in greater detail in the text.

In the figures:

FIG. 1a schematically illustrates the removal of a contaminant particle from an endoscope channel by flushing with the cleaning fluid;

FIG. 1b schematically illustrates the removal of a biofilm from an endoscope channel by flushing with the cleaning fluid;

FIG. 2 schematically illustrates a pressure profile of a cleaning fluid in an endoscope channel during a cleaning process;

FIG. 3a schematically illustrates the functioning of a diaphragm pump (suction cycle);

FIG. 3b schematically illustrates the functioning of a diaphragm pump (discharge cycle); and

FIG. 4 schematically illustrates an example of a cleaning and disinfecting device.

In the drawings, the same or similar elements and/or parts are provided with the same reference numbers in order to prevent the item from needing to be reintroduced.

### DETAILED DESCRIPTION

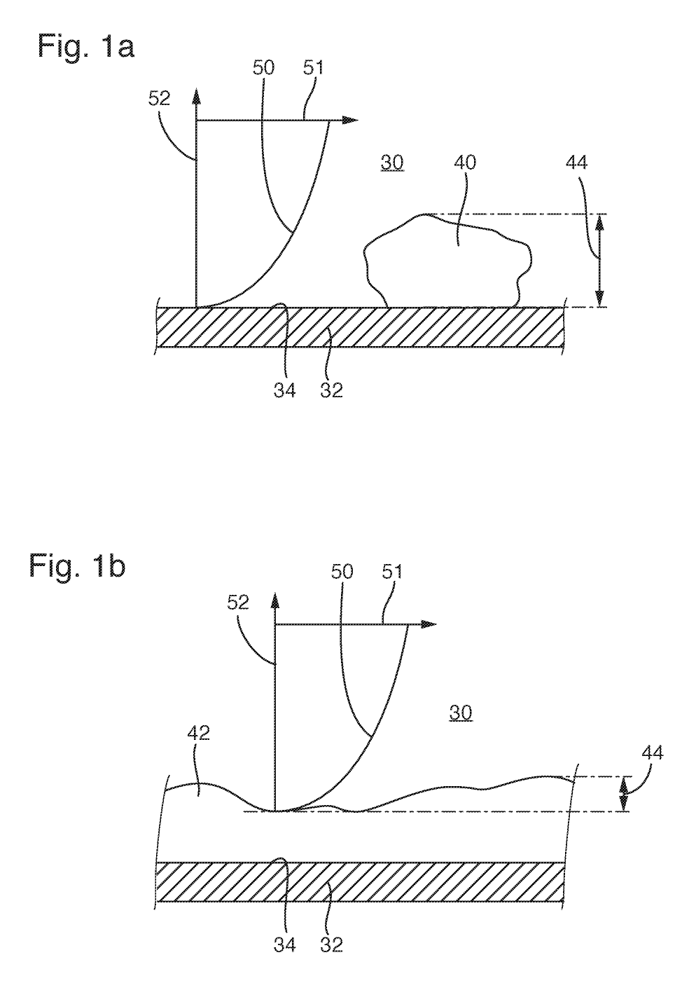

FIG. 1a and 1b schematically illustrate a section of an endoscope channel 30 which is bordered on the side by a side wall 32, which can be flexible. The side wall 32, or respectively, the endoscope channel 30, has an inner surface 34.

The endoscope channel 30 is closed in the peripheral direction, for example because the side wall 32 is configured in the shape of a hose. This is not shown in FIG. 1a for reasons of clarity.

In FIG. 1a, a contaminant particle 40 adheres to the inner surface 34 of the endoscope channel 30 which is to be removed by flushing the endoscope channel 30 with a cleaning fluid. A flow profile 50 of the cleaning fluid is also schematically illustrated in FIG. 1a which represents the flow speed 51 of the cleaning fluid depending on the distance 52 from the inner surface 34 at a randomly selected point in the longitudinal direction of the endoscope channel 30 at a randomly selected time.

The contaminant particle 40 has an effective contact surface 44 upon which the flow of cleaning fluid acts. Shearing forces are thereby exerted on the contaminant particle 40.

When the flow of cleaning fluid contacts the surface 34 of the endoscope channel 30 in front of the contaminant particle 40, a boundary layer between the contaminant particle 40 and surface 34 is stressed by the shearing forces. This causes the contaminant particle 40 to release from the surface 34, wherein the released contaminant particle 40 is carried away with the flow of cleaning fluid and flushed out of the endoscope channel.

The greater the shearing forces, the faster and more completely the contaminant particle 40 is released from the inner surface 34 of the side wall 32, wherein the shearing forces are further reinforced when a swirling of the cleaning fluid occurs at the contaminant particle 40.

FIG. 1b schematically illustrates the endoscope channel 30 from FIG. 1a with contamination in the form of a biofilm 42. The biofilm 42 is an extensive, closed layer of contamination on the inner surface 34 of the side wall 32 of the endoscope channel 30 which for example has a viscous to slimy consistency.

The biofilm 42 has a much smaller effective contact surface 44 than an isolated contaminant particle 40, and a substantially laminar flow flows over it. The effective contact surface 44 does not extend to the inner surface 34 of the side wall 32 and is limited to the top layers of the biofilm 42 at a distance from the surface 34.

Consequently, only the surface of the biofilm 42 is contacted by cleaning fluid at a constant pressure and volumetric flow in conventional flushing, and is at best removed in layers, and is therefore insufficiently removed, or only after a very long time of flushing.

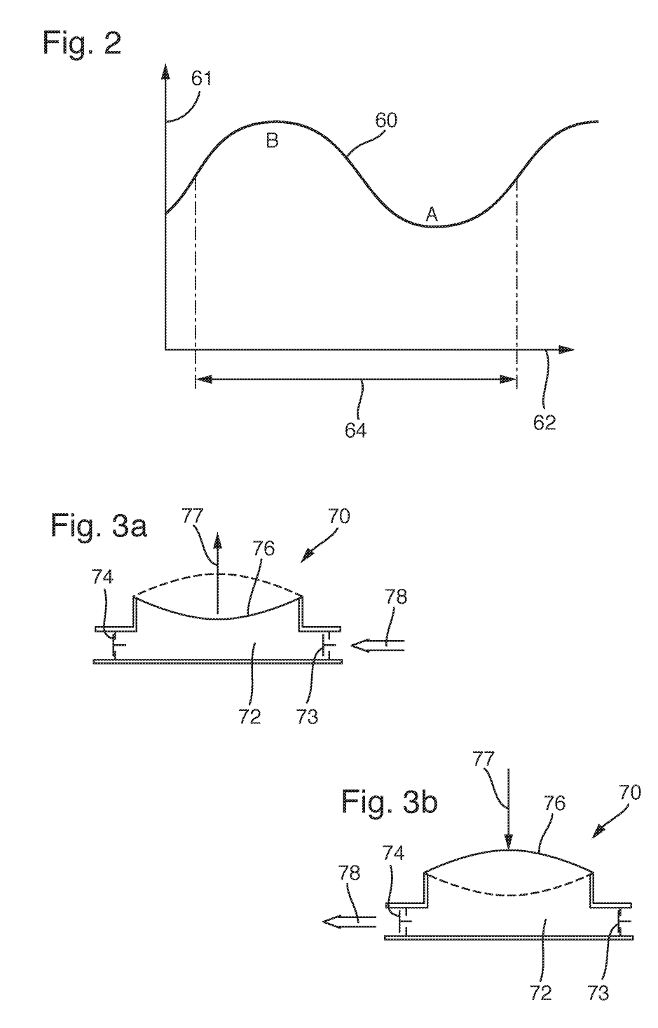

A varying pressure is applied to the cleaning fluid in the endoscope channel 30. An example of a pressure profile 60 is schematically illustrated in FIG. 2. The pressure profile 60 presents a pressure 61 of the cleaning fluid in the endoscope channel depending on the time 62.

The pressure profile 60 has for example recurring periods 64 which each comprise a low pressure phase or a low pressure cycle A and a high-pressure phase or a high pressure cycle B. The pressure 61 which varies over time of the cleaning fluid in the endoscope channel 30 leads to elastic deformations, or deformations of the side wall 32 of the endoscope channel 30, which can be a flexible side wall. The endoscope channel 30 expands when a low-pressure phase B switches to a high-pressure phase A, and contracts when a high-pressure phase B switches to a low pressure phase A.

The difference in pressure between a high pressure phase and a low-pressure phase can be set such that the direction of flow of the flushing agent in the endoscope channel 30 is always retained when switching between the high-pressure phase to the low-pressure phase and vice versa.

The repeated deformations of the side wall 32 cause a significantly faster and more complete release of the biofilm 42 from the surface 34 of the endoscope channel 30 than would be possible solely due to the shearing forces acting on the biofilm by means of the flowing cleaning fluid.

This can be achieved by any pressure profile 60 with pressure 61 changing over time, such as by individual pressure surges at an irregular sequence over time as well.

The pressure profile 60 can be realized by means of a diaphragm pump 70.

FIGS. 3a and 3b schematically illustrate the functioning of a diaphragm pump 70.

The diaphragm pump 70 comprises a housing with a pump chamber 72 which has an inlet with an inlet valve 73, and an outlet with an outlet valve 74. A side wall of the pump chamber 72 can be configured as a flexible or mobile diaphragm 76 which can be moved from outside of the pump chamber 72 by means of an actuator (not shown). The actuator can act mechanically, pneumatically or hydraulically on the diaphragm 76.

FIG. 3a shows a first work cycle of the diaphragm pump 70 during which cleaning fluid is drawn into the pump chamber 72. For this purpose, the diaphragm 76 shown as a continuous line is moved by the actuator along the arrow 77 into the position shown as a dashed line. During the suction or suction cycle, the inlet valve is open to enable an inflow of the cleaning fluid through the inlet depicted by the arrow 78. At the same time, the outlet valve 74 is closed to prevent a backflow of cleaning fluid through the outlet.

FIG. 3b shows a second work cycle of the diaphragm pump 70 during which cleaning fluid in the pump chamber 72 is discharged under pressure through the outlet. For this purpose, the diaphragm 76 shown as a continuous line is moved by the actuator along the arrow 77 into the position shown as a dashed line. During the discharge or discharge cycle, the outlet valve 74 is open to enable an outflow of the cleaning fluid through the outlet depicted by the arrow 78. At the same time, the inlet valve 73 is closed to prevent a backflow of cleaning fluid through the inlet.

If the cleaning fluid is already under pressure at the inlet or pressure is applied upstream from the inlet, this pressure counteracts the backflow against the direction of flow 78 when drawing and discharging. In this case, the inlet valve 73 and/or the outlet valve 74 can be optionally discarded.

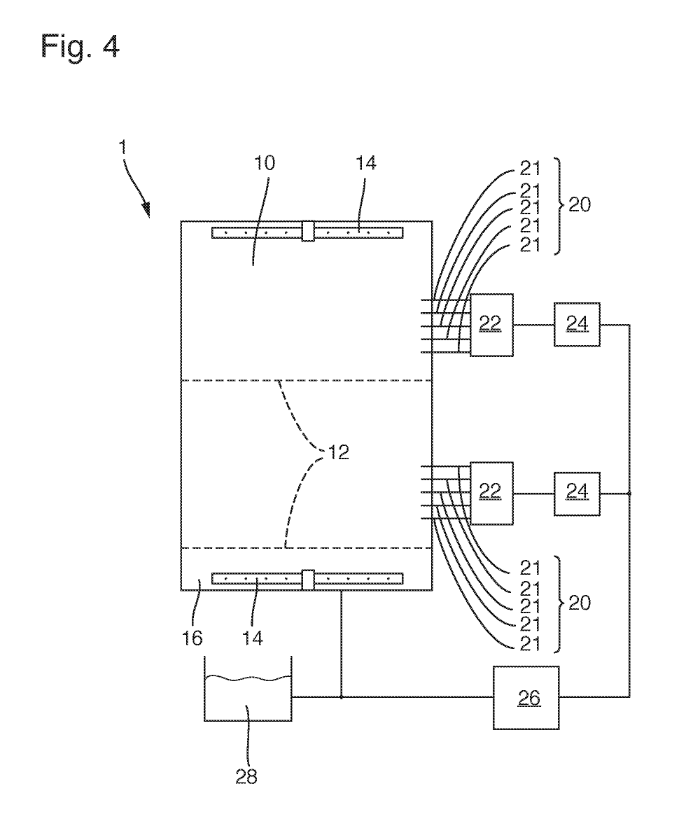

FIG. 4 shows an example of a cleaning and disinfecting device 1 for cleaning endoscopes. The cleaning and disinfecting device 1 comprises a flushing chamber 10 with two flushing levels 12 which can each comprise a wire basket, a perforated plate or a comparable, fluid-permeable resting surface for an endoscope.

In the flushing chamber 10, two spray devices 14 are provided for cleaning the outside of endoscopes placed on the flushing levels 12. The spray devices 14 have suitable feed lines for fluid cleaning and/or disinfecting means which are not shown for reasons of clarity.

To clean endoscope channels 30 of endoscopes placed on the flushing levels 12, each flushing level 12 is assigned a connection group 20 that each has a plurality of connections 21 for one endoscope channel 30 in each case. One endoscope channel 30 is connected to a connection 21 in each case and is flushed with cleaning fluid through the connection 21.

The depicted number of five connections 21 per connection group 20 is to be understood expressly as an example, more or less than five connections 21 per connection group 20 and/or a different number of connections can be provided for the two connection groups 20.

For each connection group 20, a control device 22 is provided which is supplied with cleaning fluid through a connection line to clean or flush the endoscope channels 30 connected to the connections 21 of the respective connection group 20.

The cleaning fluid can originate from a storage tank 28 and passes through a system with a plurality of pumps 24, 26 to the two control devices 22.

The control fluid is distributed by means of the control devices 22 to the different connections 21 of the respective connection group 20, wherein individual connections 21 can be configured to to be able to be shut off in the event that only part of the connections 21 of the relevant connection group 20 is needed to clean one or more endoscopes.

The pump system 24, 26 comprises a booster pump 24 configured as a diaphragm pump 70 for each of the supply devices 22, as well as a common circulating pump 26 configured as a reciprocating pump.

The circulating pump 26 can be used to pump cleaning fluid through the described line system and any endoscope channels 30 connected to the connections 21. For this purpose, a sufficiently high, even pressure is applied to the cleaning fluid, such as by means of the circulating pump 26.

The booster pumps 24 are connected over the course of cleaning corresponding to the method described above in order to vary or modulate the delivery pressure provided by the circulating pump 26. The variation or modulation of the pressure level can be separately connected or set for each of the connection groups 20 since each connection group 20 is assigned its own booster pump 24.

The cleaning fluid guided through the connections 21 into an endoscope channel flows through the endoscope channel and reaches the flushing chamber 10 at its open end opposite the connection 21. There, under the influence of gravity, the cleaning fluid flows or drips through the permeable flushing levels 12 into the bottom area of the flushing chamber 10 which can be configured as a reservoir 16. The cleaning fluid can be drained from the reservoir 16 and discarded, or as shown in FIG. 4, can be fed in a closed circuit to the circulating pump 26 to be reused.

While there has been shown and described what is considered to be preferred embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

### LIST OF REFERENCE NUMBERS

1 Cleaning and disinfecting device
10 flushing chamber
12 flushing level
14 Spray device
16 Reservoir
20 Connection group
21 Connection
22 Control device
24 Booster pump
26 Circulating pump
28 Storage tank
30 Endoscope channel
32 Side wall
34 Inner surface
40 Contaminant particle
42 Biofilm
44 Effective contact surface
50 Flow profile
51 Flow speed
52 Distance
60 Pressure profile
62 Pressure
63 Time
64 Period
70 Diaphragm pump
72 Pump chamber
73 Inlet valve
74 Outlet valve
76 Diaphragm
77 Membrane movement
78 Flow rate
A Low-pressure cycle
B High-pressure cycle

What is claimed is:

1. A method for cleaning a surgical instrument with a cleaning fluid, the surgical instrument having at least one channel, the method comprising:
applying a pressure to the cleaning fluid,
flushing the channel by means of the cleaning fluid, and
varying the pressure during the flushing.

2. The method according to claim 1, wherein the varying of the pressure is repeated during the flushing.

3. The method according to claim 1, wherien the varying of the pressure is repeated at regular intervals over time.

4. The method according to claim 1, further comprising checking a cleanlinessof the channel.

5. The method of claim 4, wherien the checking comprises one or more of determining a flow rate and determining a pressure drop within the channel.

6. The method of claim 5, wherein a constant pressure is applied to the cleaning fluid during the checking.

7. A device for cleaning a surgical instrument having at least one channel by means of a cleaning fluid, the device comprising:
a pump device for the cleaning fluid; and
a connection for the channel for flushing the channel with the cleaning fluid,
wherein the pump device is configured to apply a varying pressure to the cleaning fluid while flushing the channel.

8. The device according to claim 7, wherein the pump apparatus comprises a cyclical pump device.

9. The device accorindg to claim 8, wherein the cyclical pump comprises a diaphragm pump.

10. The device according to claim 7, wherein the pump device comprises a continuous pump device.

11. The device of claim 10, wherein the continuous pump device comprises an automatic control device for changing an output pressure of the continuous pump device.

12. The device according to claim 7, wherein the pump device comprises at least two pump devices.

13. The device according to claim 12, wherein one of the at least two pump devices comprises a cyclical pump device and another of the at least two pump devices comprises a continuous pump device.

14. The device according to claim 7, wherein the at least one channel comrpises two or more channels and the device further comprising a connection group having a plurality of connections for each of the two or more channels.

15. The device according to one claim 14, wherein the pump device assigned exclusively to the connection group.

16. The device according to one claim 7, wherein the pump device is assigned exclusively to the connection.

17. The device according to claim 7, further comprising a control device for checking cleaning of the channel connectable to the connection

18. The device according to claim 17, wherein the control device is configured to determine onr or more of a flow rate andr a pressure drop in the channel.

19. A method of using a cyclical pump device and a continuous pump device for cleaning a channel of a surgical instrument.

20. The method of claim 19, wherein the cyclical pump comprises a diaphragm pump and the continuous pump device comprises a reciprocating pump.
